(19) United States
(12) Patent Application Publication
(10) Pub. No.: US 2025/0359926 A1
(43) Pub. Date: Nov. 27, 2025

(54) MULTIFUNCTIONAL PULSE ENERGIZING DEVICE AND PROCESSING METHOD FOR THE SAME

(71) Applicant: SHENZHEN PULSECARE MEDICAL TECHNOLOGY CO., LTD., Shenzhen (CN)

(72) Inventor: Jianwen Tan, Shenzhen (CN)

(21) Appl. No.: 19/260,316

(22) Filed: Jul. 4, 2025

Related U.S. Application Data
(63) Continuation of application No. PCT/CN2025/087188, filed on Apr. 3, 2025.

(30) Foreign Application Priority Data
May 27, 2024 (CN) 202410662242.0
Sep. 6, 2024 (WO) PCT/CN2024/117514

(51) Int. Cl. A61B 18/14; A61B 17/00; A61B 18/00; A61B 18/16; A61M 25/00; A61M 25/01

(52) U.S. Cl. CPC A61B 18/1492; A61B 18/16; A61M 25/0015; A61B 2017/00526; A61B 2018/00023; A61B 2018/00267; A61B 2018/00351; A61B 2018/00577; A61B 2018/00613; A61B 2018/00702; A61B 2018/1467; A61B 2018/162; A61M 25/0147

(57) ABSTRACT

A multifunctional pulse energizing device includes a multi-cavity component and a first pull component. A portion of the multi-cavity component between a proximal end and a distal end is cut along an axial direction into a plurality of sub-tube portions; the multi-cavity component is provided with a multi-layer structure, which is configured to form the sub-tube portions and a central cavity; and a plurality of first electrodes are disposed on the sub-tube portions respectively, a second electrode is disposed on the central component, and a third electrode is disposed at a distal end of the multi-cavity component. The multifunctional pulse energizing device has the functions of single-point ablation and annular ablation to tissues, with diverse functions, simple process and low difficulty in production and manufacturing.

### CROSS-REFERENCE TO RELATED APPLICATIONS

The present disclosure is a continuation application of PCT Application No. PCT/CN2025/087188, filed on Apr. 3, 2025, which claims the priorities of Chinese Patent Application No. 202410662242.0, filed on May 27, 2024; and PCT Application No. PCT/CN2024/117514, filed on Sep. 6, 2024, the entire contents of which are hereby incorporated by reference.

### TECHNICAL FIELD

The present disclosure relates to the technical field of medical devices, and particularly to a multifunctional pulse energizing device and a processing method for the same.

### BACKGROUND

Atrial fibrillation is the most common arrhythmia, which may lead to stroke, cardiomyopathy, and the like, and may lead to death in severe cases. With the increase in age, the incidence of atrial fibrillation continues to rise. Percutaneous catheter ablation is a first-line treatment for atrial fibrillation and has been widely recognized. A purpose of ablation is to destroy potential arrhythmic myocardial tissue, prevent the propagation of abnormal electrical signals, or destroy the abnormal electrical signal conduction of cardiac tissue. Ablation therapy includes a plurality of aspects: one aspect is thermal ablation, such as radiofrequency ablation, laser ablation, microwave ablation, and the like, and the other aspect is pulsed ablation using the principle of bioelectroporation.

### SUMMARY

In some embodiments, the present disclosure provides a multifunctional pulse energizing device, including: a multi-cavity component defining a central cavity and a plurality of peripheral cavities surrounding the central cavity therein; the plurality of peripheral cavities and the central cavity extending along an axial direction without communicating with each other; wherein a portion of the multi-cavity component between a proximal end and a distal end in the axial direction is cut along the axial direction into a plurality of sub-tube portions that are separated in a circumferential direction of the multi-cavity component; each of the plurality of sub-tube portions is provided with a respective peripheral cavity therein, and each of the plurality of sub-tube portions is provided with a through hole communicating with the respective peripheral cavity; at least a portion of the multi-cavity component from the plurality of sub-tube portions to the distal end is provided with a multi-layer structure; and the multi-layer structure comprises a cutting layer and an inner layer, the cutting layer is configured to form the plurality of sub-tube portions, and the inner layer is configured to form a central component; a plurality of first electrodes, each of the first electrodes being mounted around a corresponding sub-tube portion and configured to deliver a pulse current; a second electrode disposed at a distal end of the central component, and configured to perform single-point discharge; and a third electrode, disposed at a distal end of multi-cavity component and configured to perform single-point discharge.

The present disclosure further provides a processing method for processing the multifunctional pulse energizing device described above, and the processing method includes: inserting a positioning pin into the central cavity of a multi-cavity component to be processed, and inserting a plurality of core rods into the plurality of peripheral cavities; placing and fixing the multi-cavity component on a processing tooling, and bringing a cutting blade on the processing tooling to abut against the portion of the multi-cavity component provided with the multi-layer structure; driving the multi-cavity component or the cutting blade on the processing tooling to move, causing the cutting blade to cut the multi-cavity component to form the plurality of the sub-tube portions; and removing the multi-cavity component from the processing tooling, and pulling out the positioning pin and each core rod respectively, wherein when the cutting blade abuts against a portion to be cut on the multi-cavity component, the cutting blade is inserted into the cutting layer without contacting the inner layer.

### BRIEF DESCRIPTION OF DRAWINGS

In order to more clearly illustrate the technical solutions in the embodiments of the present disclosure, a brief introduction to drawings described in the embodiments is as follows. Apparently, the following drawings are merely some embodiments of the present disclosure, for those skilled in the art, other drawings can be obtained based on these drawings without creative labor.

FIG. 1 is a structural schematic view of a multifunctional pulse energizing device according to one embodiment of the present disclosure.

FIG. 2-1 illustrates a state of a multifunctional pulse energizing device according to one embodiment of the present disclosure.

FIG. 2-2 is a structural schematic view of a multifunctional pulse energizing device according to some embodiments of the present disclosure.

FIG. 2-3 is a structural schematic view of a multifunctional pulse energizing device according to some other embodiments of the present disclosure.

FIG. 3 is a structural schematic view of a multifunctional pulse energizing device according to one embodiment of the present disclosure, in which a central component is removed.

FIG. 4 is an operation schematic view of a multifunctional pulse energizing device according to one embodiment of the present disclosure, with sub-tube portions in a bent state.

FIG. 5 is a schematic view of a multifunctional pulse energizing device according to one embodiment of the present disclosure, with sub-tube portions restored to an initial state.

FIG. 6 is a cross-sectional schematic view of a multi-cavity component according to one embodiment of the present disclosure.

FIG. 7 is a cross-sectional schematic view of an insulated electrical lead according to one embodiment of the present disclosure.

FIG. 8 is a cross-sectional schematic view of the central component according to one embodiment of the present disclosure.

FIG. 9 is a cross-sectional schematic view of an electrode according to one embodiment of the present disclosure.

FIG. 10 is a schematic view of an electrode provided with a voltage balancing structure according to one embodiment of the present disclosure.

FIG. 11 is a schematic view of an electrode provided with a voltage balancing ring according to one embodiment of the present disclosure.

FIG. 12 is a structural schematic view of a processing tooling according to one embodiment of the present disclosure.

FIG. 13 is a schematic view of a multi-cavity component inserted with a positioning pin and core rods according to one embodiment of the present disclosure.

### DESCRIPTION OF EMBODIMENTS

In order to make the purpose, technical solutions and advantages of the present disclosure more clearly understood, the present disclosure is further described in detail below in conjunction with accompanying drawings and embodiments. It should be understood that specific embodiments described herein are only used to explain the present disclosure and are not intended to limit the present disclosure.

Various specific technical features described in the various embodiments can be combined in any appropriate manner as long as there is no contradiction. For example, different embodiments and technical solutions can be formed by combining different specific technical features. In order to avoid unnecessary repetition, various possible combinations of the specific technical features in the present disclosure are not described separately.

In the following descriptions, the terms “first, second . . . ” are only used to distinguish different objects, and do not indicate that the objects have similarities or connections with each other. It should be noted that the orientation descriptions “above”, “below”, “outside”, “inside” and the like mentioned herein are all orientations in a normal use state. The “left” and “right” directions refer to the left and right directions shown in the corresponding schematic views, which may be or may not be the left and right directions in the normal use state.

It should be noted that the terms “include”, “comprise” or any other variants thereof are intended to cover a non-exclusive inclusion, therefore, a process, method, article or device that includes/comprises a series of elements is not limited to those elements, but also includes/comprises other elements not explicitly listed, or elements inherent to such process, method, article or device. Without further limitations, an element defined by the phrase “including/comprising one . . . ” does not exclude the presence of other identical elements in the process, method, article or device including/comprising the element. “Plurality” means a number greater than or equal to two.

Pulsed Field Ablation (PFA) is a novel tissue ablation method based on high-voltage pulsed energy, which has emerged in recent years and mainly uses a principle of irreversible electroporation (IRE). Through the action of a high-voltage pulsed electric field on cells, irreversible perforations are generated in cell membranes, thereby causing the cells to gradually necrose, to ultimately achieve a purpose of tissue ablation. Due to differences in tissue electrical properties and damage thresholds of cells to high-voltage pulsed energy, PFA exhibits excellent tissue selectivity. For example, myocardial tissue is more sensitive to high-voltage pulsed electric fields, while neural tissue has a higher tolerance to pulsed electric fields. Therefore, by selecting appropriate intensity of the high-voltage pulsed electric field, ablation of selective tissue, such as ablation of tissue near nerves and blood vessels, and the like, can be achieved. Except for the selective tissue mentioned above, PFA is generally considered a non-thermal ablation technique. That is, no heat and no temperature increase in tissue occurs during an ablation process, thereby avoiding the heat sink effect associated with traditional ablation methods such as radiofrequency, microwave, and cryoablation through irreversible electroporation. Therefore, PFA is considered to have superior advantages in ablation of temperature-sensitive tissues (such as tissues in proximity to the gallbladder, bile duct, esophagus and the like), especially when atrial fibrillation is treated by ablation, PFA has the advantages of short ablation duration and protection of tissues such as the treatment area or blood vessels.

When PFA ablation for atrial fibrillation is performed, for some paroxysmal atrial fibrillation ablation and all persistent atrial fibrillation ablation, not only circumferential ablation of pulmonary veins is required, but also ablation of the posterior wall or roof of the left atrium is required. Therefore, a PFA catheter used should be able to perform not only circumferential tissue ablation but also single-point tissue ablation. However, typical PFA catheters have a single function and cannot meet different usage requirements simultaneously. Moreover, the structures of typical PFA catheters are complicated, and the production and processing are difficult.

Embodiments of the present disclosure provide a multifunctional pulse energizing device or catheter, and the multifunctional pulse energizing device or catheter is usually connected to an operating device and a high-voltage pulse generator. The purpose of tissue ablation is achieved by means of inserting one end of the multifunctional pulse energizing device into a blood vessel, and advancing the multifunctional pulse energizing device along the blood vessel into a tissue to be treated by operating the operating device (such as the control handle), delivering a high-voltage, high-frequency pulse voltage generated by the high-voltage pulse generator to the multifunctional pulse energizing device after the multifunctional pulse energizing device has been delivered in place, thereby establishing a high-intensity electric field at the tissue to be treated, and forming an area with high current density, through the action of the high-voltage pulsed electric field on cells, irreversible perforations being generated in the cell membrane, thereby causing the cells to gradually undergo necrosis.

The term “electroporation” herein refers to the application of an electric field to cell membranes to change the permeability of the cell membranes to an extracellular environment. The term “irreversible electroporation” herein refers to the application of an electric field to cell membranes to permanently change the permeability of the cell membranes to the extracellular environment. For example, it can be observed that one or more pores are formed in the cell membrane when the cell is subjected to irreversible electroporation, and the one or more pores remain after the electric field is removed. The term “proximal end 101” herein refers to one end of the multifunctional pulse energizing device that is adjacent to an operator, or one end that is connected to an action of the operator. The term “distal end 102” herein refers to one end of the multifunctional pulse energizing device that is inserted into the blood vessel, or one end that is adjacent to the tissue to be treated.

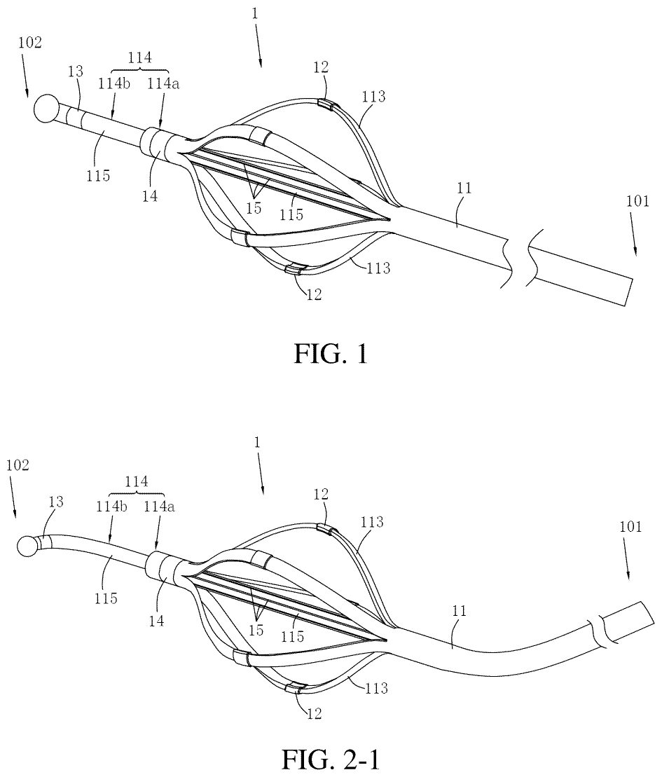

As shown in FIG. 1 to FIG. 5, a multifunctional pulse energizing device 1 is provided by one embodiment of the present disclosure, including a multi-cavity component 11 and a plurality of first electrodes 12. The multi-cavity component 11 is generally a tubular material with a circular shaped cross-section, and is provided with a central cavity 111 and a plurality of peripheral cavities 112 surrounding the central cavity 111 therein. The peripheral cavities 112 and the central cavity 111 extend along an axial direction of the multi-cavity component 11 and are non-communicating with each other. For example, both a length direction of each peripheral cavity 112 and a length direction of the central cavity 111 extend substantially along a length direction of the multi-cavity component 11.

In some other embodiments, a wall of the multi-cavity component 11 has a thickness greater than a preset value, for example, 0.5 mm to 1.5 mm. The specific value of the thickness is appropriate to ensure a reliable configuration of the central cavity 111 and each peripheral cavity 112, without causing an overall diameter to become excessively large, which is not conducive to intravascular delivery.

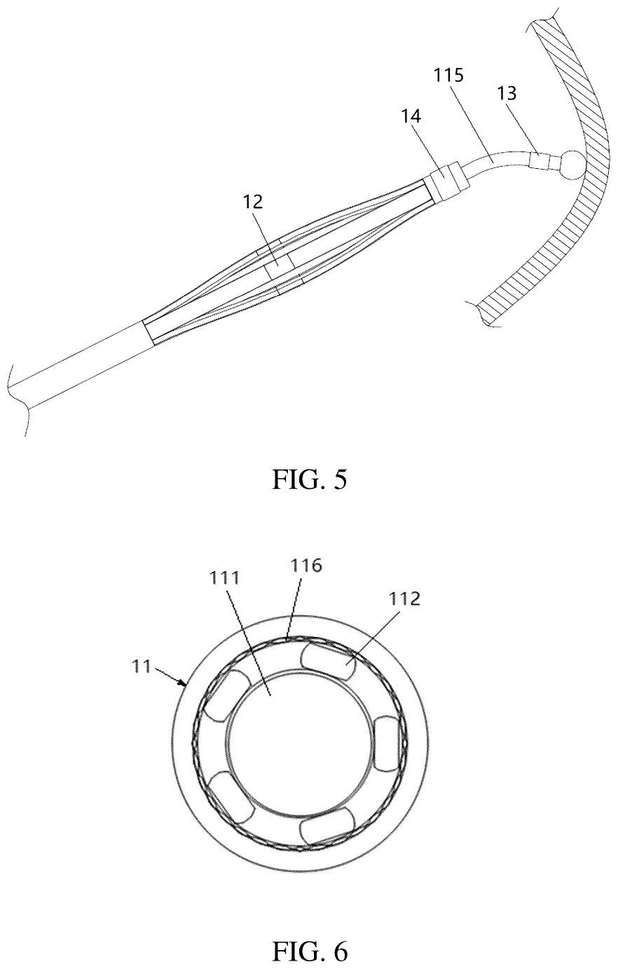

In some embodiments, a portion of the multi-cavity component 11 between a proximal end and a distal end in the axial direction is cut into a plurality of sub-tube portions 113 along the axial direction, and the sub-tube portions 113 are arranged to separate apart in a circumferential direction of the multi-cavity component 11. A length direction of each sub-tube portion 113 is arranged along the length direction of the multi-cavity component 11. Each sub-tube portion 113 is provided with one peripheral cavity 112 therein. A distal end of a cutting layer 114a is axially movable, so that each sub-tube portion 113 can be transformed between one configuration of extending along a straight line and another configuration of protruding outward in a curved shape. For example, as shown in FIG. 4, each sub-tube portion 113 possesses elastic deformability and can be bent under the action of an external force, and after the external force disappears, as shown in FIG. 5, each sub-tube portion 113 can be collapsed under the action of an elastic deformation force of each sub-tube portion 113 itself, and restored to an initial state or substantially initial state. Generally, the multi-cavity component 11 is made of a polymer material meeting medical usage standards, such as Pebax (thermoplastic nylon elastomer) and PA (polyamide), which have high strength, high fracture resistance, and excellent elasticity. The multi-cavity component 11 can be formed into a structure with different hardness at different positions by gradual transition. For example, the hardness gradually decreases from the distal end to the proximal end, so that the distal end has better insertion performance, which facilitates guiding movement in the blood vessel, while the hardness gradually decreases as approaching the proximal end, which is convenient for bending adjustment. The hardness of a middle region of the multi-cavity component 11 is moderate, for example, the hardness of the middle region of the multi-cavity component 11 is greater than the hardness of a proximal end of the multi-cavity component 11, to ensure the overall strength and pushability. Of course, the hardness of the multi-cavity component 11 can also be set to other distributions according to usage requirements, for example, the hardness of a certain region is designed to be higher or lower, which are flexible in design flexibility is excellent.

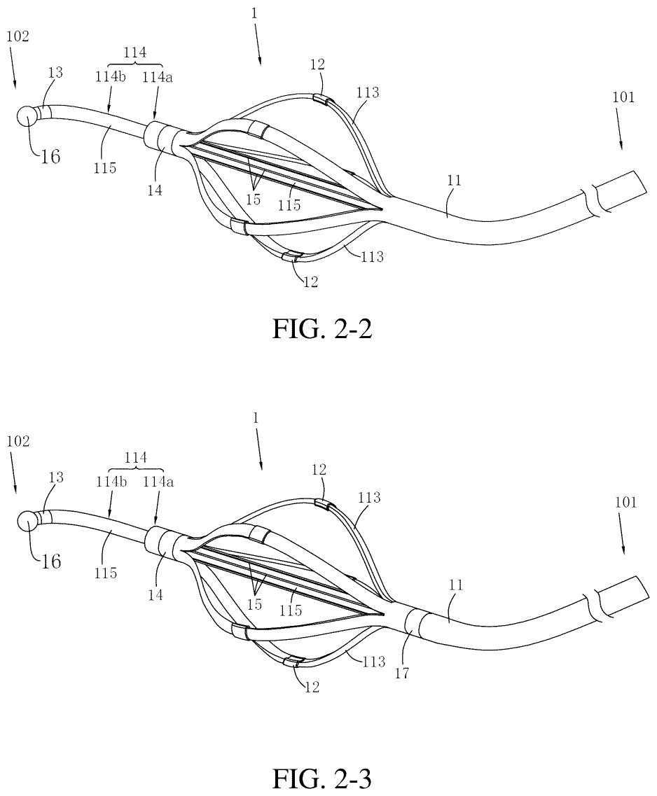

In some embodiments, as shown in FIG. 1 and FIG. 2-1, at least a portion of the multi-cavity component 11 from each sub-tube portion 113 to the distal end is provided with a multi-layer structure 114. The multi-layer structure 114 includes a cutting layer 114a and an inner layer 114b. The cutting layer 114a is configured to form the sub-tube portions 113, and the inner layer 114b is configured to form a central component 115. That is, a portion of the multi-cavity component 11 used to form the sub-tube portions 113 and a region from the portion to the distal end are configured to include at least the cutting layer 114a and the inner layer 114b. Along a radial direction of the multi-cavity component 11, each sub-tube portion 113 is located outside the central component 115, and the central component 115 can provide a guide path for the bending deformation of each sub-tube portion 113. For example, the bending deformation of each sub-tube portion 113 includes deformation from a first configuration (such as a straight extension state) to a second configuration (such as a cage shape) or other configurations (such as other shape configurations other than the straight extension state and the cage shape, or shape configurations intermediate between the straight extension state and the cage shape). In this configuration, the sub-tube portions 113 and the central component 115 can be formed through processing one multi-cavity component 11, without excessive additional structural designs, thereby greatly improving the convenience of manufacturing the multifunctional pulse energizing device 1. Moreover, by adjusting a position of each sub-tube portion 113 on the central component 115, a deformation degree of each sub-tube portion 113 can be adjusted, thereby adjusting a degree of contact between each sub-tube portion 113 and the tissue to achieve a better ablation effect.

In some other embodiments, the central component 115 (or the inner layer 114b) is slidably or movably engaged with the cutting layer 114a, forming each sub-tube portion 113. That is, the central component 115 is slidably inserted into the central cavity 111 of the multi-cavity component 11 (or the cutting layer 114a).

In some embodiments, as shown in FIG. 2-1 and FIG. 3, each sub-tube portion 113 is provided with a first electrode 12. For example, the first electrode 12 is mounted around a corresponding sub-tube portion 113, and configured to deliver a pulse current into the multifunctional pulse energizing device 1. For example, the pulse current is delivered in a state where each sub-tube portion 113 is protruding outward in the curved shape or in a state where each sub-tube portion 113 is extended along the straight line.

In some embodiments, the first electrodes 12 disposed on the sub-tube portions 113 are substantially maintained on a same concentric ring in the circumferential direction of the multi-cavity component 11, thereby achieving the annular ablation of tissue through the joint action of the first electrodes 12 at different positions in the circumferential direction of the multi-cavity component 11. For example, as shown in FIG. 4, when the sub-tube portions 113 are bent into the cage shape, adjacent first electrodes 12 can be discharged in sequence, forming a full circle of annular ablation finally, thereby achieving the annular ablation of tissue. The expression “substantially maintained on a same concentric ring” means that cross-sections obtained at a center point of the first electrodes 12 perpendicular to the axis of the multi-cavity component 11 are on a same circle, or within an allowable error range, so that the first electrodes 12 can achieve the annular ablation of tissue at a same circumferential position.

In some embodiments, as shown in FIG. 1 and FIG. 2-1, a second electrode 13 is disposed at a distal end of the central component 115, and the number of the second electrode 13 is exemplarily one.

In some embodiments, a third electrode 14 is disposed at a distal end of the cutting layer 114a. In some other embodiments, the second electrode member 13 may be used as a mapping electrode.

In some other embodiments, as shown in FIG. 2-2, the distal end of the central component 115 is provided with a head end electrode 16, and the head end electrode 16 is located at a distal end of the second electrode 13. That is, the second electrode 13 is closer to the sub-tube portions 113 than the head end electrode 16.

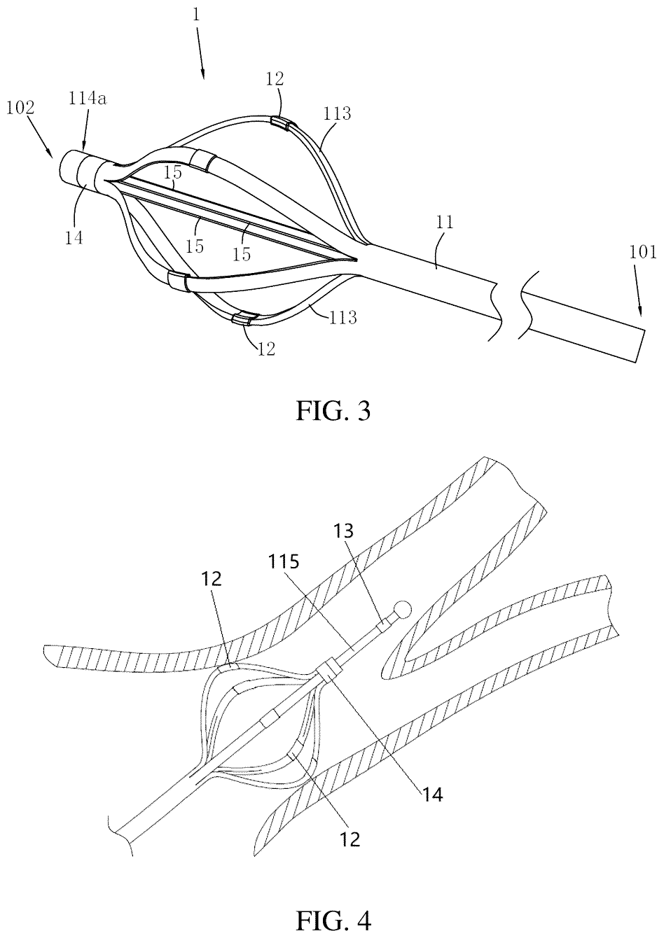

In some other embodiments, as shown in FIG. 2-3, the multi-cavity component 11 is provided with a return electrode 17, and the return electrode 17 is located at a proximal side of the sub-tube portions 113. That is, the return electrode 17 is located on the multi-cavity component 11 adjacent to the sub-tube portions 113.

In this way, single-point ablation of tissue can be achieved by delivering a pulse current into the second electrode 13, the third electrode 14, or the head end electrode 16. Thus, functionality is enhanced, thereby meeting usage requirements for the single-point ablation of tissue.

Optionally, the single-point ablation may be achieved by discharge between the second electrode 13 and the third electrode 14. Alternatively, the single-point ablation in a smaller range may be achieved by discharge through the second electrode 13. The expressions “the distal end of the central component 115” and “the distal end of the connected sub-tube portions 113” refer to an end that is farther from the operator in distance, or when inserted into the blood vessel, an end that first enters the blood vessel.

Alternatively, the single-point ablation may be achieved by discharging between the head end electrode 16 and the third electrode 14, or the single-point ablation in a smaller range may be achieved by discharging through the head end electrode 16.

Alternatively, the single-point ablation may be achieved by discharging between the head end electrode 16 and the return electrode 17, or the single-point ablation in a smaller range may be achieved by discharging through the head end electrode 16.

Alternatively, the single-point ablation may be achieved by discharging between the third electrode 14 and the return electrode 17, or the single-point ablation in a smaller range may be achieved by discharging through the third electrode 14.

In some other embodiments, as shown in FIG. 2-1 and FIG. 3, the multifunctional pulse energizing device 1 further includes one or more first pull components 15. One end of the first pull component 15 is inserted into the central cavity 111 (refer to FIG. 6), and extends to connect with the third electrode 14 and/or a distal end of the multi-cavity component 11. Alternatively, one end of the first pull component 15 is inserted into the central cavity 111 (refer to FIG. 6), and extends to connect with the third electrode 14 and/or the distal end of the cutting layer 114a. The other opposite end of the first pull component 15 is connected to the operating device or used for hand-held operation by the operator. In this way, an acting force can be transmitted to the distal end of the cutting layer 114a or the third electrode 14 by pulling the first pull component 15, causing the distal end of the cutting layer 114a to move axially along the central component 115 (in other words, the distal end of the cutting layer 114a is slidably or movably mounted around the outer periphery of the central component 115). Therefore, the sub-tube portions 113 are transformed between the two configurations of extending along the straight line and protruding outward in the curved shape.

For example, by connecting one end of the first pull component 15 to the distal end of the cutting layer 114a, the distal end of the cutting layer 114a can be pulled to slide reciprocally on the central component 115. Alternatively, since the third electrode 14 is disposed at the distal end of the cutting layer 114a, by connecting one end of the first pull component 15 to the third electrode 14, the distal end of the cutting layer 114a can also be pulled to slide reciprocally on the central component 115. Of course, one end of the first pull component 15 can also be connected to both the distal end of the cutting layer 114a and the third electrode 14, so that the distal end of the cutting layer 114a can be pulled to slide reciprocally on the central component 115. In this way, when the sub-tube portions 113 are pulled by the first pull component 15 into a state of protruding outward in the curved shape or extending along the straight line, a pulse current is introduced into the first electrodes 12, thereby achieving a therapeutic purpose of the annular ablation of tissue.

In some other embodiments, when the sub-tube portions 113 are maintained in a straight extension state, the second electrode 13 is covered, and the single-point ablation of tissue can be achieved by delivering the pulse current into the third electrode 14 or the head end electrode 16. When a curved degree of each sub-tube portion 113 gradually decreases and is about to become the straight extension state, the second electrode 13, the third electrode 14 or the head end electrode 16 can be used individually or simultaneously for the single-point ablation of tissue, thereby achieving a flexible use mode.

In some cases, under high-voltage and high-frequency pulses (for example, nanosecond pulses or millisecond pulses), the first electrodes 12 may be easy to contact with blood and generate a certain amount of heat, surface temperatures of the first electrodes 12 increase to promote a coagulation mechanism of the blood around the first electrodes 12, thereby resulting in scabs on surfaces of the first electrodes 12. The scabs further increase the contact resistance between the first electrodes 12 and the blood, resulting in a vicious cycle.

In some embodiments of the present disclosure, a through hole (not shown in the figures) communicating with the peripheral cavities 112 is defined on each sub-tube portion 113. The through hole is arranged adjacent to the first electrodes 12, and can be located at any position around a contour shape of the first electrodes 12. For example, the number of the through holes is more than one, such as 2 to 6, and a plurality of through holes are spaced apart along a circumferential direction of the sub-tube portions 113.

Fluid (such as saline) is injected into each peripheral cavity 112, and then the injected fluid flows out from a corresponding through hole. Fluid flowing out from the vicinity of the first electrodes 12 not only improves the electrical conductivity of a surrounding area, but also plays a role in continuously cooling the first electrodes 12, thereby achieving the purpose of cooling the first electrodes 12 and reducing the risk of scab formation. Of course, it is understandable that through holes for fluid passage can also be provided adjacent to the second electrode 13 and the third electrode 14, cooling the second electrode 13 and the third electrode 14 and reducing the risk of scab formation on the second electrode 13 and the third electrode 14, thereby improving the safety and reliability of treatment. Alternatively, the through holes for fluid passage may also be provided adjacent to the second electrode 13, the third electrode 14, the head end electrode 16, and/or the return electrode 17.

In some embodiments, the through hole may be circle, rectangular, ellipse, and the like, and is optionally configured as a circle. A diameter of the through hole is exemplarily in a range of 0.05 mm to 0.5 mm. For example, the diameter of the through hole may be 0.05 mm, 0.06 mm, 0.1 mm, 0.13 mm, 0.21 mm, 0.29 mm, 0.32 mm, 0.4 mm, 0.47 mm, 0.5 mm, and the like. Of course, the diameter of the through hole may also be any other value in the range of 0.05 mm to 0.5 mm.

The multifunctional pulse energizing device 1 provided by the embodiment of the present disclosure includes the multi-cavity component 11 and the first electrodes 12. The multi-cavity component 11 is provided with the central cavity 111 and the peripheral cavities 112 surrounding the central cavity 111 therein. By axially cutting the portion of the multi-cavity component 11 between the proximal end and the distal end, the sub-tube portions 113 separated in the circumferential direction are formed, each sub-tube portion 113 is provided with one peripheral cavity 112 therein, and each sub-tube portion 113 is provided with the first electrodes 12, thereby achieving the annular ablation treatment of tissue. Moreover, a portion of the multi-cavity component 11 extending from each sub-tube portion 113 to the distal end is configured as a multi-layer structure 114, which is used to form the sub-tube portions 113 and the central component 115; and the second electrode 13 is disposed at the distal end of the central component 115, the third electrode 14 is disposed at the distal end of the cutting layer 114a. Therefore, the single-point ablation treatment of tissue can be achieved by the second electrode 13 and/or the third electrode 14. In one embodiment of the present disclosure, the distal end of the cutting layer 114a is pulled by the first pull component 15 to move axially along the central component 115, thereby driving the sub-tube portions 113 to transform between two configurations of extending along the straight line and protruding outward in the curved shape. Therefore, treatment modes can be switched between single-point ablation and annular ablation of tissue, and the treatment modes are diversified.

In some embodiments, the multifunctional pulse energizing device 1 is directly processed on the basis of the multi-cavity component 11, and the multi-cavity component 11 itself has a simple structure that meets usage requirements. Therefore, too many other complicated structural designs are not required, resulting in a simpler structure of the multifunctional pulse energizing device 1. In addition, the processing and production are achieved through cutting. The production process is relatively simple, has low requirements, and can be reproduced, thereby well meeting the reproducibility requirements of the multifunctional pulse energizing device 1.

In some other embodiments, as shown in FIG. 1 and FIG. 2-1, the number of the sub-tube portions 113 formed by cutting the multi-cavity component 11 can be more than one, optionally 3 to 12, and the specific number of the sub-tube portions 113 may be set according to actual usage requirements. In this way, in the same position, the sub-tube portions 113 are oriented toward different positions in the circumferential direction, so that discharge ablation can be performed on different parts of the tissue at the location without the need of rotation in the same position to achieve treatment of different parts, thereby improving the convenience of treatment and reducing the discomfort of patients during treatment. Moreover, each sub-tube portion 113 formed by cutting is relatively independent and has excellent deformation performance, thereby enhancing the fit with the first pull component 15 and the fit among the sub-tube portions 113, which facilitate reducing an overall outer diameter of the multi-cavity component 11. For example, the overall outer diameter of the multi-cavity component 11 can be within 3.2 mm, thereby improving the passability in the blood vessel and meeting requirements for use in smaller blood vessels.

In some other embodiments, as shown in FIG. 1 and FIG. 2-1, the number of the first electrode 12 disposed on each sub-tube portion 113 is exemplarily one, so that a treatment position of the first electrode 12 can be controlled well. Of course, it is understandable that in other embodiments, the number of the first electrodes 12 disposed on each sub-tube portion 113 may be two or more, to achieve a purpose of adjusting a treatment range. For example, the first electrodes 12 disposed on the sub-tube portion 113 may be slipped onto the sub-tube portion 113 from a tip of the distal end thereof, and then moved and adjusted to a set position on the sub-tube portions 113; or may be formed by wrapping around the set position on the sub-tube portions 113, thereby providing flexible arrangement methods. When two or more first electrodes 12 are disposed on each sub-tube portion 113, the through hole for injecting salt water can be disposed between two first electrodes 12 and closer to the first electrode 12 at the distal end.

In some embodiments, as shown in FIG. 3 and FIG. 6, the first pull component 15 may be a metal wire, such as stainless steel, nickel titanium, or the like. The number of the metal wires can be set according to usage requirements, for example, the number of the metal wires is 2 to 4. FIG. 3 exemplarily shows three first pull components 15. The number of the first pull components is not limited herein. One end of the metal wire is inserted from the central cavity 111 and extends to the distal end of the cutting layer 114a, to connect to the third electrode 14 or the distal end of the cutting layer 114a, or simultaneously connect to both the third electrode 14 and the distal end of the cutting layer 114a. The first pull component 15 is movable relative to the central component 115, thereby driving the distal end to move, so that the sub-tube portions 113 can be transformed as needed between states of protruding outward in the curved shape and in the cage shape. In addition, the metal wire such as stainless steel or nickel titanium can be coated with a PTFE (polytetrafluoroethylene) or PI (polyimide) coating, thereby improving the durability of the metal wire and making the metal wire safer; thus, meeting requirements of medical use.

In some embodiments, length directions of the 2 to 4 first pull components 15 extend along a longitudinal axis direction of the multi-cavity component 11. The 2 to 4 first pull components 15 are arranged close to each other or spaced apart from each other. For example, the 2 to 4 first pull components 15 are arranged side-by-side in a plane parallel to the longitudinal axis of the multi-cavity component 11, and the side-by-side arrangement can be closely attached to each other or arranged at intervals. Alternatively, the 2 to 4 first pull components 15 are spaced apart along the circumferential direction of the multi-cavity component 11. Alternatively, along a cross-sectional direction of the multi-cavity component 11, the 2 to 4 first pull components 15 are located at different cross section positions. For example, when the number of the first pull components 15 is at least three, a cross section defined by the at least three first pull components 15 is perpendicular to the length direction of the multi-cavity component 11.

It can be understandable that the length directions of 2 to 4 first pull components 15 extend along the longitudinal axis direction of the multi-cavity component 11, which does not merely include that any part of the first pull component 15 in the length direction extends along the longitudinal axis of the multi-cavity component 11, but also include that at least a part of the first pull component 15 in the length direction extends along the longitudinal axis direction of the multi-cavity component 11 (for example, the length direction of the part of the first pull component 15 adjacent to the sub-tube portions 113 extends along the longitudinal axis direction of the multi-cavity component 11), and at least a part of the first pull component 15 in the length direction may be deviated from the central axis of the multi-cavity component 11 and be fixed to other structures (for example, an operating handle).

In some embodiments, as shown in FIG. 1 and FIG. 6, each first electrode 12 is connected to a first insulated electrical lead (not shown in the figures) disposed in peripheral cavity 112, through which a current is delivered to the first electrode 12. For example, an effective diameter of the first insulated electrical lead is not less than 0.12 mm, and an overall diameter of an ultra-fine multi-layer insulated electrical lead does not exceed 0.25 mm. The insulation breakdown strength of the first insulated electrical lead is exemplarily more than 5 kV. Through a multi-layer insulation structure, the risk of electromagnetic interference generated during corona discharge is reduced, and the risk of short circuit caused by conduction during saline perfusion is also reduced. Moreover, a length of each sub-tube portion 113 is exemplarily set to 30 mm to 80 mm, so that the length of each sub-tube portion 113 is within an appropriate range. When in the curved shape, the sub-tube portions 113 may have a diameter that meets use requirements, thereby avoiding insufficient working area due to insufficient length or insufficient structural strength due to overlength.

In some embodiments, as shown in FIG. 6 and FIG. 8, the central component 115 is provided with a plurality of sub-cavities 1151 therein, and the sub-cavities 1151 extend axially and communicate with the central cavity 111. At least one of the sub-cavities 1151 is provided with a second pull component (not shown in the figures) for bending the central component 115; and/or, at least another one of the sub-cavities 1151 is provided with a second insulated electrical lead (not shown in the figures) for connecting to the second electrode 13 (refer to FIG. 1). Optionally, the number of the sub-cavities 1151 is generally exemplarily set to more than 3, and the sub-cavities 1151 are evenly spaced and distributed in the circumferential direction. During an actual treatment process, the central component 115 is generally inserted into the blood vessel first. Since the blood vessel has diverse shapes and a plurality of bends, the central component 115 is required to follow the plurality of bends during an advancing process to improve pushing smoothness. Therefore, by providing the sub-cavities 1151 inside the central component 115, and then providing a movable second pull component in any one of the sub-cavities 1151, one end of the second pull component is connected to the distal end of the central component 115, and the other end extends toward the operator, so that the second pull component can be connected to the operating device or pulled by the operator to obtain a pulling force, thereby adjusting a shape of the distal end of the central component 115 and improving the pushing smoothness. Moreover, at least one of remaining sub-cavities 1151 is provided with the second insulated electrical lead for connecting to the second electrode 13, so that a pulse current can be delivered to the second electrode 13. Other remaining sub-cavities 1151 can be used as a fluid (such as salt water) perfusion channels, for injecting fluid (such as saline) which flows out from a position in proximity to the second electrode 13 through the corresponding through hole, thereby achieving a purpose of cooling the second electrode 13 and reducing the risk of scab formation. Of course, the other remaining sub-cavities 1151 may also be used for reserved functional expansion.

In some embodiments, a position of the multi-cavity component 11 (for example, the distal end or the proximal end of the multi-cavity component 11) adjacent to each sub-tube portion 113 is also set as an adjustable bending structure, so that a shape of the multi-cavity component 11 can be adjusted as needed during pushing, thereby improving the convenience of controlling the multi-cavity component 11.

In some embodiments, the third electrode 14 is disposed at the distal end of the cutting layer 114a. In order to achieve delivery of a pulse current to the third electrode 14, the third electrode 14 may be connected to a third insulated electrical lead (not shown in the figures). The third insulated electrical lead is passed through the peripheral cavity 112 in any one of the sub-tube portions 113 to connect with the high-voltage pulse generator; thus, a radial size of the multi-cavity component is not increased, and the ingenuity of design is excellent.

In some embodiments, in order to achieve delivery of a pulse current to the head end electrode 16, the head end electrode 16 may be connected to a fourth insulated electrical lead (not shown in the figures). The fourth insulated electrical lead passes through the peripheral cavity 112 in any one of the sub-tube portions 113 to connect with the high-voltage pulse generator; thus, the radial size of the multi-cavity component 11 is not increased, and the ingenuity of design is excellent.

In some embodiments, in order to achieve delivery of a pulse current to the return electrode 17, the return electrode 17 may be connected to a fifth insulated electrical lead (not shown in the figures). The fifth insulated electrical lead passes through the peripheral cavity 112 in any one of the sub-tube portions 113 to connect with the high-voltage pulse generator; thus, the radial size of the multi-cavity component 11 is not increased, and the ingenuity of design is excellent.

In some embodiments, a structure of each of the fourth insulated electrical lead and the fifth insulated electrical lead is identical with a structure of the first insulated electrical lead, the second insulated electrical lead, or the third insulated electrical lead.

In some embodiments, the multifunctional pulse energizing device 1 further includes a balloon (not shown in the figures), the balloon is configured to limit a movement position of the distal end of the cutting layer 114a on the central component 115. The balloon is mounted around the central component 115, and an inner cavity of the balloon is communicated with one of remaining sub-cavities 1151, so that a filling medium can be introduced into the balloon through the sub-cavity 1151. For example, the balloon is made of a thin membrane that can be expanded or collapsed elastically. The balloon is mounted around the central component 115 and disposed between the distal end of the cutting layer 114a and the central component 115 (such as the distal end of the central component 115). Thus, when the balloon is expanded, a gap between the distal end of the cutting layer 114a and the central component 115 (such as the distal end of the central component 115) can be filled, thereby achieving the positioning of the distal end of the cutting layer 114a on the central component 115, so as to position the curved state of each sub-tube portion 113. Therefore, the requirements for adjusting the curved shapes of the sub-tube portions 113 under different treatment modes are met, the arrangement is ingenious, thereby greatly improving the treatment means of the multifunctional pulse electrification device 1. The filling medium may be gas, liquid, or the like.

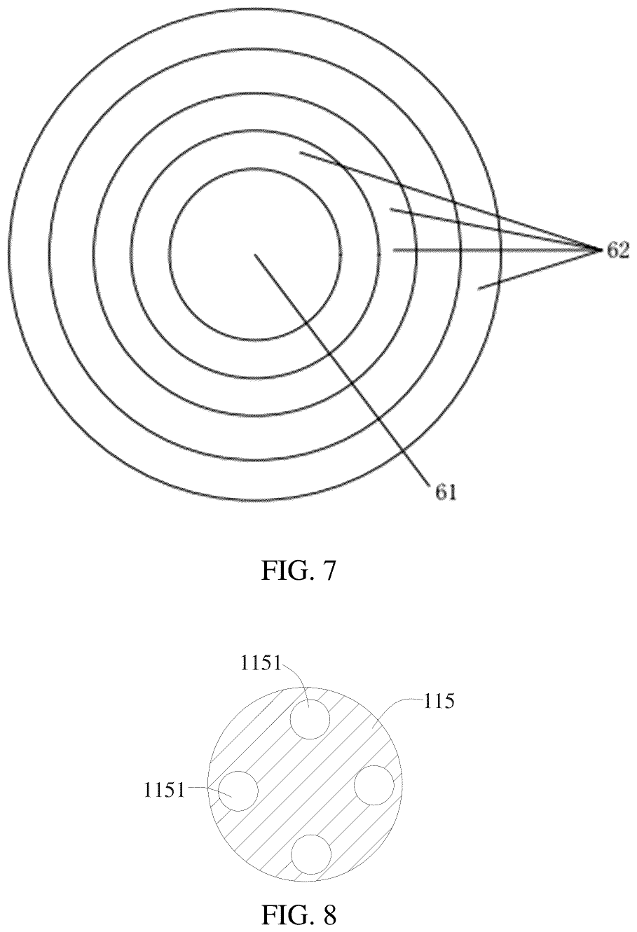

In some embodiments, as shown in FIG. 7, each of the first insulated electrical lead and the second insulated electrical lead includes a conductive core 61 and an insulating layer 62. The conductive core 61 is connected to the corresponding first electrode 12 or the second electrode 13. In one embodiment, the insulating layer 62 is configured as multi-layer structure, and the multiple layers of the insulating layers 62 are wrapped on the conductive core 61 layer by layer and extend along a length direction of the conductive core 61. Each insulating layer 62 is coaxially arranged with the conductive core 61. For example, the conductive core 61 may be composed of a copper core, and a diameter is exemplary not less than 0.12 mm. A PTFE (polytetrafluoroethylene) or PI (polyimide) coating can be used for insulation treatment among ultra-fine copper wires composing the copper core, thereby improving the insulation ability of the conductive core 61.

In one embodiment of the present disclosure, the insulating layer 62 is optionally configured with four layers, under the premise of meeting the overall insulation performance requirements, an overall size of the insulated electrical leads can allow the insulated electrical leads to pass through the corresponding peripheral cavity 112 (refer to FIG. 3). It should be understood that, the structure of the third insulated electrical lead is identical with the structure of each of the first insulated electrical lead and the second insulated electrical lead. The third insulated electrical lead also includes the conductive core 61 and the insulating layer 62. The conductive core 61 is connected to the third electrode 14. In one embodiment, the insulating layer 62 is configured as multi-layer structure, and the multiple layers of the insulating layer 62 are wrapped on the conductive core 61 layer by layer and extend along a length direction of the conductive core 61. In this way, there is no need to separately select materials for the third insulated electrical lead, thereby improving the convenience of production.

In some embodiments, during a PFA (pulsed electric field ablation) surgery, intracardiac potential signals need to be mapped to achieve electrophysiological examination and immediate efficacy assessment of ablation therapy. Mapping is carried out by amplifying and acquiring weak cardiac electrical signals, while ablation requires the release of high-voltage pulse energy through the electrodes. In order to prevent the high-voltage pulse from damaging a detection circuit of the cardiac electrical signals. In one embodiment of the present disclosure, each first electrode 12 is connected to individual lead wire, and an insulation voltage between any two first electrodes 12 can exemplarily reach above 5 kV, thereby integrating mapping and ablation functions, and achieving multiplexing of the ablation and mapping functions through fast switching in a host system.

In some embodiments, as shown in FIG. 1 and FIG. 6, a braided layer 116 is provided in the multi-cavity component 11. The braided layer 116 extends from the proximal end of the multi-cavity component 11 toward the sub-tube portions 113, approaching the sub-tube portions 113 but does not extend into a region of the sub-tube portions 113. In a radial direction, the braided layer 116 is disposed on an outer side of each peripheral cavity 112. For example, in the radial direction, for a region of each peripheral cavity 112 located on a proximal side of the sub-tube portions 113, the braided layer 116 is disposed on the outer side of each peripheral cavity 112.

By providing the braided layer 116, a torque transmission capability of the multi-cavity component 11 is improved, thereby achieving reliable movement of the whole component in the blood vessel. The braided layer 116 may be a stainless steel braided mesh, exhibiting high strength and suitable elastic bending deformation performance. The braided layer 116 may be disposed in the multi-cavity component 11 in a discontinuous way in the circumferential direction, instead, a plurality of braided layers 116 are respectively arranged at outsides of the peripheral cavities 112, and the braided layers 116 are not connected with each other. In this way, the strength of the peripheral cavities 112 is enhanced, and the overall structural strength is improved. In addition, since the distal end of the cutting layer 114a needs to be cut to form the sub-tube portions 113, the braided layer 116 does not extend into the region of the sub-tube portions 113, avoiding affecting the cutting process of the multi-cavity component 11.

In some embodiments, as shown in FIG. 1 and FIG. 6, the braided layer 116 may be arranged by surrounding the multi-cavity component 11 in the circumferential direction, with each peripheral cavity 112 is located in a region surrounded by the braided layer 116. In this configuration, in the circumferential direction of the multi-cavity component 11, the outer sides of the sub-tube portions 113 are surrounded by the same braided layer 116, which simultaneously provides protection for the sub-tube portions 113 to avoid accidental puncture.

In some embodiments, to achieve adjustment of the hardness of the multi-cavity component 11 as needed, it may be achieved by changing a material forming the multi-cavity component 11, and it may also be achieved by changing a thickness or a density of the braided layer 116, diverse setting ways are allowed.

In some embodiments, in a direction perpendicular to the length of the multi-cavity component 11, cross-sectional shapes of the peripheral cavities 112 are at least partially identical. That is, the cross-sectional shapes of the peripheral cavities 112 obtained in a same reference direction are at least partially identical. The insulated electrical leads extend out of the peripheral cavities 112. In this way, there is no need to selectively insert the insulated electrical leads due to different shapes of the peripheral cavities 112, thereby improving the convenience of installation. Moreover, an inner wall surface of each peripheral cavity 112 is configured as a smooth curved surface with a small friction resistance, which is conducive to increase the smoothness of moving the insulated electrical leads, and also facilitates the flow of fluids (such as saline). For example, it may provide the inner wall surface of each peripheral cavity 112 as the smooth curved surface by a processing technology, or by providing a PTFE (polytetrafluoroethylene) lining layer. Similarly, the inner wall surface of the central cavity 111 may also be provided with the PTFE (polytetrafluoroethylene) lining layer to improve the surface smoothness.

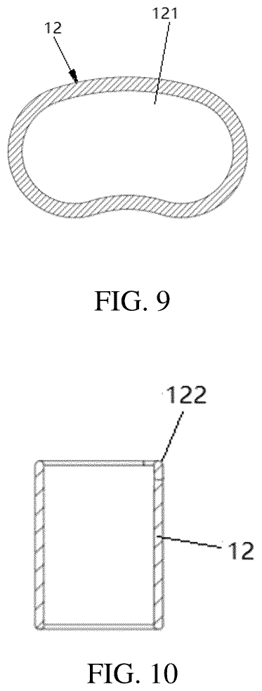

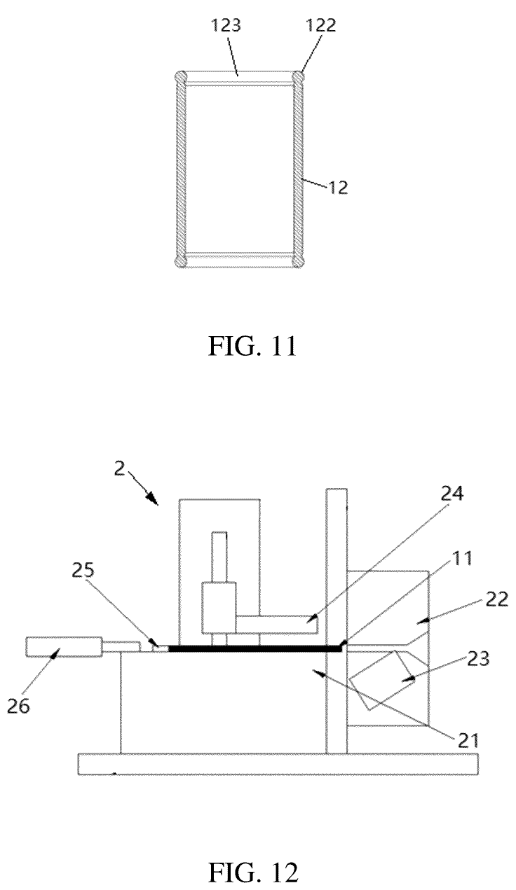

In some embodiments, as shown in FIG. 9 to FIG. 11, a receiving hole 121 is defined in each first electrode 12 (refer to FIG. 1), and the receiving hole 121 is used to be inserted with the sub-tube portions 113, so that the first electrodes 12 can be mounted on the sub-tube portions 113. In addition, a discharge side of each end of the first electrodes 12 is provided with a voltage balancing structure 122. Alternatively, the discharge side of each end of the first electrodes 12 is connected with a voltage balancing ring 123, and the voltage balancing structure 122 is provided on the voltage balancing ring 123. For example, since the multifunctional pulse energizing device 1 needs to withstand a higher voltage, during pulsed electric field ablation surgery, the high-voltage pulse energy is discharged through electrodes on the catheter. When the high-voltage pulse is discharged, in order to prevent tip discharge or spark discharge on the electrodes, the electric field distribution is designed to be more uniform. In some embodiments of the present disclosure, by providing the voltage balancing structures 122 or the voltage balancing rings 123 having the voltage balancing structures 122 on the discharge sides at both ends of the first electrodes 12, and the voltage balancing structure 122 is provided with a smooth circular arc curved surface and the voltage balancing structures 122 provided with smooth circular arc curved surfaces, the discharge sides at both ends of the first electrodes 12 are free of tips due to the voltage balancing structures 122. Therefore, the electric field distribution is more uniform, and no serious power line distortion point is formed at both ends of the first electrodes 12, avoiding or reducing the spark discharge caused by the tips of the first electrodes 12 when a high-voltage nanosecond pulse exemplarily reaching 10 kV, thereby improving the safety and service life.

In some embodiments, a discharge side of each end of the second electrode 13 and a discharge side of each end of the third electrode 14 may also be provided with the voltage balancing structure 122 or the voltage balancing ring 123 having the voltage balancing structure 122, as such, the spark discharge caused by the tips of the second electrode 13 and the third electrode 14 during operation can be avoided or reduced, thereby improving the safety and service life.

In some embodiments, as shown in FIG. 1 and FIG. 9, in a direction perpendicular to the length of the sub-tube portions 113, at least a cross-sectional shape of the receiving hole 121 is identical to cross-sectional outer contour shapes of the sub-tube portions 113, and the first electrodes 12 are conforming to outer walls of the sub-tube portions 113. In this way, after the first electrodes 12 are installed on the sub-tube portions 113, the first electrodes 12 can be closely conformed to outer surfaces of the sub-tube portions 113, which not only achieves discharge reliably, but also forms no local protrusion, thereby reducing the overall radial dimension of the multi-cavity component 11. In addition, in this way, cross-sectional outer contour shapes of the first electrodes 12 may be identical to or different from the cross-sectional shape of the receiving hole 121, and this configuration can be adjusted as needed.

In some embodiments, in the direction perpendicular to the length of the sub-tube portions 113, the cross-sectional outer contour shapes of the first electrodes 12 are identical to the cross-sectional outer edge shapes of the sub-tube portions 113. This configuration can ensure that cross-sectional shapes of the multi-cavity component 11 perpendicular to the axial direction remain consistent and are all circular, the overall aesthetics are excellent, and it is also convenient to be delivered through the blood vessel.

In some embodiments, a positioning sensor (not shown in the figures) for positioning is disposed on at least one of the sub-tube portions 113, and the positioning sensor is adjacent to the proximal end of the multi-cavity component 11. In this way, positions of the sub-tube portions 113 can be sensed by the positioning sensor, thereby achieving accurate treatment. Optionally, the positioning sensor is a magnetic positioning sensor, which has excellent positioning effect and is safe in operation.

The multifunctional pulse energizing device 1 provided by the embodiment of the present disclosure is provided with the multi-cavity component 11, which can be processed directly on the basis of the multi-cavity component 11 without requiring too many other complicated structural designs, resulting in a simpler structure of the multifunctional pulse energizing device 1. The processing and production are achieved through cutting. The production process is relatively simple with low requirements, and can be reproduced repeatedly, which excellently meets the reproducibility requirements of the multifunctional pulse energizing device 1.

In some embodiments of present disclosure, by providing the first electrodes 12 on each sub-tube portion 113, annular ablation treatment to the tissues can be achieved. By configuring the portion of the multi-cavity component 11 extending from the sub-tube portions 113 to the distal end as the multi-layer structure 114, the sub-tube portions 113 and the central component 115 can be formed; and the second electrode 13 is disposed at the distal end of the central component 115, the third electrode 14 is disposed at the distal end of the cutting layer 114a, single-point ablation treatment of tissue can be achieved by the second electrode 13 and/or the third electrode 14. Through a multi-layer insulation design of the insulated electrical lead, the insulation capacity between first electrodes 12 is improved, so that insulation levels of different first electrodes 12 can meet nanosecond pulse discharge requirements with higher voltage and higher repetition frequency, thereby preventing the generation of corona discharge and interfering electrical signals. Through the design of the voltage balancing structures 122 at end surfaces of each electrode, the electric field distribution is more uniform, thereby effectively preventing the generation of spark discharge and greatly reducing the heat generation during the discharge process. Through a form-fitting design between the first electrodes 12 and the sub-tube portions 113, the overall outer diameter of the multi-cavity component 11 is effectively reduced, thereby improving the overall deliverability and passability. In addition, each first electrode 12 is connected to a separate insulated electrical lead, and separate insulated electrical leads are insulated from each other, thereby realizing the time-sharing multiplexing of the first electrodes 12. Therefore, during the PFA surgery, the ablation and mapping can be performed at different periods based on the same first electrode 12. Therefore, during PFA surgery, ablation and mapping can be performed at different periods based on the same first electrode 12.

In some embodiments of the present disclosure, a processing method is further provided to process the multifunctional pulse energizing device 1. As shown in FIG. 12, the processing method employs a processing tooling 2 to process the multi-cavity component 11. The processing tooling 2 includes a workbench 21, a blade holder 22 mounted on the workbench 21, a cutting blade 23 mounted on the blade holder 22, a positioning block 24 configured to position the multi-cavity component 11, a positioning seat 25 configured to abut against the multi-cavity component 11, and a push rod 26 configured to push the positioning seat 25. The processing method includes steps as follows.

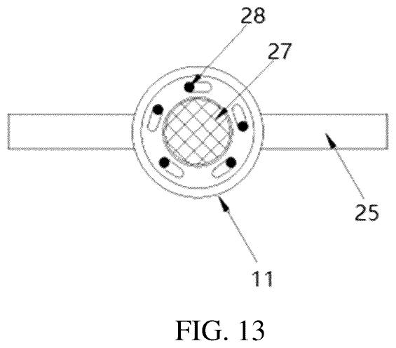

As shown in FIG. 12 and FIG. 13, a positioning pin 27 is inserted into the central cavity 111 of a multi-cavity component 11 to be processed, and a core rod 28 is inserted into a respective peripheral cavity 112. The positioning pin 27 and the core rod 28 are configured to improve the overall hardness of the multi-cavity component 11 during processing, thereby preventing deformation during processing. Then, the multi-cavity component 11 is placed on the workbench 21 of the processing tooling 2 with one end abutting against the positioning seat 25 and the middle position of the multi-cavity component 11 being pressed by the positioning block 24 for positioning to prevent the multi-cavity component 11 from warping during the moving cutting. After the multi-cavity member 11 is positioned, the cutting blade 23 on the processing tooling 2 is brought into abut against a part of the multi-cavity component 11 having the multi-layer structure 114. After that, the positioning seat 25 is driven to move by a driving force applied by the push rod 26, causing the multi-cavity component 11 to move toward the cutting blade 23, so that the multi-cavity component 11 is cut by the cutting blade 23, to form the sub-tube portions 113.

Certainly, in some other embodiments, the multi-cavity component 11 may also be prevented from moving by the push rod 26 abutting against the positioning seat 25, and then the cutting blade 23 is moved toward the multi-cavity component 11 to cut, thereby forming the sub-tube portions 113. After the cutting is completed, the positioning block 24 is released, the processed multi-cavity component 11 is removed from the processing tooling 2, and the positioning pin 27 and each core rod 28 are respectively pulled out.

In some other embodiments, in the case that the cutting blade 23 abuts against a portion to be cut on the multi-cavity component 11, the cutting blade 23 is inserted into the cutting layer 114a without contacting the inner layer 114b, to prevent the inner layer 114b from being cut. During cutting the multi-cavity component 11, the number of the cutting blade 23 is equal to the number of the sub-tube portions 113 to be formed by cutting. An angle of the cutting blade 23 relative to an axial direction of the multi-cavity component 11 is adjusted, so that the cutting blade 23 is inclined at a certain angle relative to the axial direction of the multi-cavity component 11, thereby reducing a cutting resistance and the abrasion of the cutting blade 23. Moreover, an angle between the cutting blade 23 and the positioning block 24 may further be adjusted, so that a pressure is generated by a spring after the cutting blade 23 is installed on the blade holder 22, to ensure that the cutting blade 23 always abuts against a wall of the blade holder 22 by a lateral force, thereby ensuring the position accuracy of the cutting blade 23.

In some other embodiments, during cutting the multi-cavity component 11, the cutting blade 23 may continuously cut along a direction from a tip of the distal end of the multi-cavity component 11 toward the proximal end to form the sub-tube portions 113, and a cutting length is exemplarily 30 mm to 80 mm. In this way, the distal end of each sub-tube portion 113 is a free end. Then, the first electrodes 12 are mounted onto the sub-tube portions 113 respectively from the ends of the sub-tube portions 113, so that each sub-tube portion 113 is provided with the first electrodes 12. Each first electrode 12 is connected to the first insulated electrical lead. The first insulated electrical lead extends through the peripheral cavity 112 of the sub-tube portion 113 on which the first electrodes 12 are located, and then extends to the proximal end of the multi-cavity component 11 until it can be connected to the high-voltage pulse generator.

In some other embodiments, during cutting the multi-cavity component 11, the cutting blade 23 may continuously cut from a position away from the tip of the distal end by a preset distance in a direction toward the proximal end to form the sub-tube portions 113, and the cutting length is 30 mm to 80 mm. For example, in this cutting method, the cutting blade 23 does not start cutting directly from the tip of the distal end, but from a position spaced from the tip of the distal end by the preset distance, and the preset distance may be between 5 mm and 15 mm, to ensure that the distal ends of the sub-tube portions 113 formed after cutting are not dispersed. Certainly, the preset distance may also be other ranges. After the cutting is completed and the sub-tube portions 113 are formed, each sub-tube portion 113 is respectively wrapped with the first electrodes 12. Each first electrode 12 is connected to the first insulated electrical lead. The first insulated electrical lead 14 extends through the peripheral cavity 112 of the sub-tube portion 113 on which the first electrodes 12 are located, and then extends to the proximal end of the pulse energizing device 1 until it can be connected to the high-voltage pulse generator.

In some embodiments, after the arrangement of the first electrodes 12 is completed, the second electrode 13 is connected to the distal end of the central component 115, and the second insulated electrical lead connected to the second electrode 13 extends out from one sub-cavity 1151 inside the central component 115 to be connected to the high-voltage pulse generator. Moreover, the third electrode 14 is connected to the distal end of the cutting layer 114a, and the third insulated electrical lead connected to the third electrode 14 extends out from the peripheral cavity 112 of any sub-tube portion 113 to be connected to the high-voltage pulse generator. For example, when each sub-tube portion 113 is provided with the free end after cutting, free ends of the sub-tube portions 113 are first connected into an integrated body around the central component 115, which can reciprocate relative to the central component 115, and then the third electrode 14 is arranged.

In one embodiment, after the arrangement of the first electrodes 12 is completed, one end of the first pull component 15 is inserted into the central cavity 111 of the multi-cavity component 11 and extends to be fixedly connected to the third electrode 14 or the distal end of the cutting layer 114a to form an integrated body, or extends to be fixedly connected to both the third electrode 14 and the distal end of the cutting layer 114a to form an integrated body. Therefore, by pulling the first pull component 15, the distal end of the cutting layer 114a can be moved axially along the central component 115, to drive the sub-tube portions 113 to transform between two configurations of extending along the straight line and protruding outward in the curved shape. For example, when cutting starts from the tip of the distal end of the multi-cavity component 11, after the first electrodes 12 are disposed on the sub-tube portions 113, one end of the first pull component 15 is inserted into the central cavity 111 of the multi-cavity component 11, and then the free ends of the sub-tube portions 113 are connected to the distal end of the first pull component 15. Alternatively, the free ends of the sub-tube portions 113 are connected into an integrated body around the central component 115, and the third electrode 14 is disposed at a joint of the free ends, as such, the first pull component 15 is connected to the third electrode 14. Alternatively, the first pull component is connected to both the distal end of the cutting layer 114a and the third electrode 14, thereby completing the manufacturing process.

In some other embodiments, in the case that a manner of the cutting blade 23 starting to continuously cut from a position away from the tip of distal end of the multi-cavity component 11 by the preset distance toward the proximal end of the multifunctional pulse energizing device 1 is employed, firstly, one end of the first pull component 15 may extend through the central cavity 111 of the multi-cavity component 11 and be bonded or fused integrally with the distal end of the cutting layer 114a; then, the cutting is performed to form the sub-tube portions 113. Thus, the processing method is versatile and highly flexible.

In the processing method provided by the embodiments of the present disclosure, the positioning pin 27 is first inserted into the central cavity 111, and the core rods 28 are respectively inserted into the peripheral cavities 112; then the multi-cavity component 11 to be cut is placed on the processing tooling 2 and cut to form the sub-tube portions 113; and then, the first electrodes 12 are provided on the sub-tube portions 113 respectively, and the first pull component 15 is connected in such a manner that the distal end of the first pull component 15 is connected to the distal end of the cutting layer 114a. In this way, the production and processing of the multifunctional pulse energizing device 1 are completed. The processing method achieves processing and production by cutting, and the processing method has relatively simple processes with low requirements. Moreover, the processing method has excellent repeatability, thereby well meeting the reproducibility requirements for the multifunctional pulse energizing device 1.

The above descriptions are merely the specific embodiments of the present disclosure, but the protection scope of the present disclosure is not limited thereto. Any changes, variations, equivalent replacements, and improvements made by those skilled in the art without departing from the spirit and scope of the present disclosure, should fall into the protection scope of the claims of the present disclosure. Therefore, the protection scope of the present disclosure should be based on the protection scope of the claims.

1. A multifunctional pulse energizing device, comprising:
a multi-cavity component, defining a central cavity and a plurality of peripheral cavities surrounding the central cavity therein; the plurality of peripheral cavities and the central cavity extending along an axial direction without communicating with each other; wherein a portion of the multi-cavity component between a proximal end and a distal end in the axial direction is cut along the axial direction into a plurality of sub-tube portions that are separated in a circumferential direction of the multi-cavity component; each of the plurality of sub-tube portions is provided with a respective peripheral cavity therein, and each of the plurality of sub-tube portions is provided with a through hole communicating with the respective peripheral cavity; at least a portion of the multi-cavity component from the plurality of sub-tube portions to the distal end is provided with a multi-layer structure; and the multi-layer structure comprises a cutting layer and an inner layer, the cutting layer is configured to form the plurality of sub-tube portions, and the inner layer is configured to form a central component;
a plurality of first electrodes, each of the plurality of first electrodes being mounted around a corresponding sub-tube portion and configured to deliver a pulse current;
a second electrode disposed at a distal end of the central component, and configured to perform single-point discharge; and
a third electrode, disposed at a distal end of the cutting layer, and configured to perform single-point discharge.

2. The multifunctional pulse energizing device of claim 1, wherein the central component is provided with a head end electrode at a distal end, and the head end electrode is located at a distal side of the second electrode and configured to perform single-point discharge.

3. The multifunctional pulse energizing device of claim 1, wherein the multi-cavity component is provided with a return electrode, and the return electrode is located at a proximal side of each of the plurality of sub-tube portions and configured to perform single-point discharge.

4. The multifunctional pulse energizing device of claim 1, wherein a wall thickness of the multi-cavity component is greater than a preset value, and the preset value is 0.5 mm to 1.5 mm.

5. The multifunctional pulse energizing device of claim 1, wherein the plurality of first electrodes are configured to deliver the pulse current when the plurality of sub-tube portions are in a configuration of protruding outward in a curved shape or extending along a straight line.

6. The multifunctional pulse energizing device of claim 1, further comprising at least one first pull component, and wherein one end of the at least one first pull component is inserted into the central cavity and extends to be fixedly connected to the third electrode and/or the cutting layer; and the at least one first pull component is configured to pull the distal end of the cutting layer to move along an axial direction of the central component, to drive each of the plurality of sub-tube portions to transform between configurations of extending along a straight line and protruding outward in a curved shape.

7. The multifunctional pulse energizing device of claim 6, wherein the at least one first pull component is a metal wire; and the at least one first pull component comprises a plurality of first pull components, and a number of the plurality of first pull components is 2 to 4.

8. The multifunctional pulse energizing device of claim 1, wherein a braided layer is disposed in the multi-cavity component, and the braided layer extends from a proximal end of the multi-cavity component toward a direction approaching the plurality of sub-tube portions without extending into a region of the plurality of sub-tube portions.

9. The multifunctional pulse energizing device of claim 8, wherein in a radial direction, the braided layer is disposed on an outer side of each of the plurality of peripheral cavities.

10. The multifunctional pulse energizing device of claim 8, wherein the multi-cavity component is surrounded by the braided layer in the circumferential direction thereof, and each of the plurality of peripheral cavities is located in a region surrounded by the braided layer.

11. The multifunctional pulse energizing device of claim 8, wherein in a radial direction, for a region of each peripheral cavity located on a proximal side of the corresponding sub-tube portion, the braided layer is disposed on the outer side thereof.

12. The multifunctional pulse energizing device of claim 1, wherein in a direction perpendicular to a length of the multi-cavity component, cross-sectional shapes of the plurality of peripheral cavities are at least partially identical; and an inner wall surface of each of the plurality of peripheral cavities is a smooth curved surface.

13. The multifunctional pulse energizing device of claim 1, wherein each of the plurality of first electrodes is provided with a receiving hole for insertion of the corresponding sub-tube portion; and
wherein a discharge side of each of the plurality of first electrodes is provided with a voltage balancing structure; or a voltage balancing ring provided with the voltage balancing structure.

14. The multifunctional pulse energizing device of claim 13, wherein in a direction perpendicular to a length of the plurality of sub-tube portions, a cross-sectional shape of the receiving hole is identical to a cross-sectional outer contour shape of the corresponding sub-tube portion, and the first electrode is attached to an outer wall of the plurality of sub-tube portions.

15. The multifunctional pulse energizing device of claim 13, wherein in a direction perpendicular to a length of the plurality of sub-tube portions, a cross-sectional outer contour shape of each first electrode is identical to a cross-sectional outer contour shape of the plurality of sub-tube portions.

16. A processing method for processing the multifunctional pulse energizing device of claim 1, the processing method comprising:
inserting a positioning pin into a central cavity of a multi-cavity component to be processed, and inserting a plurality of core rods into a plurality of peripheral cavities;
placing and fixing the multi-cavity component on a processing tooling, and bringing a cutting blade on the processing tooling to abut against a portion of the multi-cavity component provided with the multi-layer structure;
driving the multi-cavity component or the cutting blade on the processing tooling to move, causing the cutting blade to cut the multi-cavity component to form the plurality of the sub-tube portions; and
removing the multi-cavity component from the processing tooling, and pulling out the positioning pin and each of the plurality of core rods respectively,
wherein when the cutting blade abuts against a portion to be cut on the multi-cavity component, the cutting blade is inserted into the cutting layer without contacting the inner layer.

17. The processing method of claim 16, wherein the cutting blade continuously cut along a direction from the distal end of the multi-cavity component toward a proximal end direction to form the plurality of sub-tube portions, and a first cutting length is 30 mm to 80 mm; or,
wherein the cutting blade continuously cut from position away from a tip of the distal end by a preset distance in a direction toward a proximal end direction to form the plurality of sub-tube portions, and a second cutting length is 30 mm to 80 mm.

18. The processing method of claim 16, wherein the distal end of the central component is provided with the second electrode, and the third electrode is connected to the distal end of the multi-cavity component.

19. The processing method of claim 18, wherein the multifunctional pulse energizing device further comprises at least one first pull component, and one end of the at least one first pull component is inserted into the central cavity of the multi-cavity component and extends to be connected to the third electrode.

20. The processing method of claim 18, wherein the multifunctional pulse energizing device further comprises at least one first pull component, and one end of the at least one first pull component is inserted into the central cavity of the multi-cavity component and extends to be connected to the distal end of the cutting layer.
